(19) United States
(12) Patent Application Publication
(10) Pub. No.: US 2022/0259624 A1
(43) Pub. Date: Aug. 18, 2022

(54) SELECTIVE PROCESS FOR PREPARING SULFOXIDES BY ENZYMATIC CATALYSIS

(71) Applicants: Arkema France, Colombes (FR); Centre National de la Recherche Scientifique (CNRS), Paris (FR); Universite d'Aix-Marseille, Marseille Cedex 7 (FR)

(72) Inventors: Georges Fremy, Lacq (FR); Hugo Brasselet, Lacq (FR); Véronique Alphand, Marseille (FR); Katia Duquesne, Vitrolles (FR)

(73) Assignees: Arkema France, Colombes (FR); Centre National de la Recherche Scientifique (CNRS), Paris (FR); Universite d'Aix-Marseille, Marseille Cedex 7 (FR)

(21) Appl. No.: 17/619,431

(22) PCT Filed: Jun. 12, 2020
(86) PCT No.: PCT/FR2020/051013

§ 371 Date: Dec. 15, 2021

(30) Foreign Application Priority Data
Jun. 17, 2019 (FR) FR1906488
Mar. 9, 2020 (FR) FR2002305

(51) Int. Cl. C12P 11/00; C12P 17/00; C12N 9/02; C12N 1/20; C12N 15/70

(52) U.S. Cl. CPC C12P 11/00; C12P 17/00; C12N 9/0073; C12N 2800/101; C12N 1/20; C12N 15/70; C12Y 114/13022

(57) ABSTRACT

The present invention relates to a selective process for preparing sulfoxides from sulfides by enzymatic catalysis, and to a composition comprising a symmetrical sulfide, an oxidoreductase enzyme catalyzing the oxidation of said symmetrical sulfide to symmetrical sulfoxide; optionally at least one cofactor C of said enzyme E; and an oxidant, which allows in particular the implementation of said process.

### CROSS-REFERENCE TO RELATED APPLICATIONS

This application is the national phase of International Application No. PCT/FR2020/051013, filed 12 Jun. 2020, which claims priority to French Application No. FR 2002305, filed 9 Mar. 2020, and French Application No. FR 1906488 filed 17 Jun. 2019, the disclosure of each of these applications being incorporated herein by reference in its entirety for all purposes.

### FIELD OF THE INVENTION

The present invention relates to a selective process for preparing organic sulfoxides from organic sulfides by enzymatic catalysis, and also to a composition enabling in particular the implementation of this process, and to uses thereof.

### BACKGROUND OF THE INVENTION

Mercaptans are of great interest industrially and are currently in very widespread use in the chemical industries, especially as starting materials in the synthesis of more complex organic molecules. For example, methyl mercaptan (CH₃SH) is used as a starting material in the synthesis of methionine, an essential amino acid used in animal nutrition. Methyl mercaptan is also used in the synthesis of dialkyl disulfides, more particularly in the synthesis of dimethyl disulfide (DMDS), a sulfiding additive for hydrotreating catalysts for petroleum fractions, among other applications.

Mercaptans, and more particularly methyl mercaptan, are generally synthesized industrially by a known process starting from alcohols and hydrogen sulfide at elevated temperature in the presence of a catalyst according to equation (1) below:

R—OH+H₂S→R—SH+H₂O (1)

However, this reaction gives rise to the formation of by-products, such as the sulfides according to equation (2) below:

R—OH+R—SH→R—S—R+H₂O (2)

Mercaptans may also be synthesized from halogenated derivatives and alkali metal, alkaline earth metal or ammonium hydrosulfides according to equation (3) below (example given using a chlorinated derivative and a sodium hydrosulfide):

R—Cl+NaSH→R—SH+NaCl (3)

This second synthesis pathway also results in the presence of unwanted sulfides.

Mercaptans, lastly, may also be synthesized from olefins and hydrogen sulfide by acid catalysis or photochemically according to whether the target is a branched or an unbranched mercaptan, according to equation (4) below:

R_{A}R_{B}C=CR_{C}R_{D}+H₂S→R_{A}R_{B}—CH—C(SH)R_{C}R_{D} (4)

Once again, this synthesis gives rise to sulfides as by-products.

These sulfides are obtained in high quantity industrially and are primarily sent for destruction. This represents an efficiency loss in the process for producing the intended mercaptan and an added cost associated with destroying them. Generation of wastes in this way is a real industrial problem for producers of mercaptans, who therefore look to derive value from these by-products. There are various ways of doing this.

First of all, there is a market for the sulfides themselves: dimethyl sulfide can be used as a food flavor or as an anticoking agent in the steam cracking of petroleum feedstocks. The demand in these markets, though, is very much lower than the amounts of sulfides produced.

The sulfides can also be converted to corresponding mercaptans by the sulfhydrolysis reaction. Nevertheless, the conditions required for carrying out this reaction are relatively harsh and give rise to new, parasitic reactions. This industrial application is therefore limited.

Finally, another means of deriving value from the sulfides produced involves the oxidation reactions of sulfides to convert them into sulfoxides and/or into sulfones. Chemical oxidation reactions of these kinds are well known. They entail different types of oxidants such as sodium hypochlorite, hydrogen peroxide, oxygen, ozone or nitrogen oxides such as N₂O₄ in the presence or absence of catalysts.

Chemical methods of oxidizing sulfides do, though, present real industrial problems. One of the most acute problems is the low chemoselectivity of the oxidation reaction. At present there is no available means industrially of guiding the oxidation exclusively toward the sulfoxides or the sulfones: a mixture of sulfoxides and sulfones is always obtained at the outcome of the oxidation of the sulfides. Other drawbacks of these chemical methods are, for example, the use of very powerful reagents, which leads to safety problems, or else the scant availability of nitrogen oxides (there are very few industrial suppliers). Some of these chemical processes, moreover, lead to problems of pollutant emission, such as the processes that use nitrogen oxides.

As well as the chemical oxidations, sulfide oxidations may be catalyzed, in biological processes, by enzymatic catalysis in solution or in organisms, generally microorganisms. These oxidations performed by enzymatic catalysis, however, are no more selective as to the products obtained; here again, a mixture of sulfoxides and sulfones is obtained from the corresponding sulfides.

Hence the low selectivity of the oxidation reaction of sulfides to sulfoxides relative to sulfones causes problems on the industrial front irrespective of the oxidation process—chemical or enzymatic—employed. It is desirable, indeed, to have the greatest possible selectivity for the product of interest, either the sulfoxides or the sulfones, for obvious reasons of cost and of quality.

The work by Bordewick et al. proposes the use of Yarrowia monooxygenases A-H for catalyzing sulfoxidation reactions of asymmetric aromatic sulfides (S. Bordewick, Enzyme Microb. Technol., 2018, 109, 31-42). In this publication, the use of techniques of genetic mutation for obtaining variants of the starting enzyme reduces the production of dimethyl sulfone by close to 95%.

The use of such techniques of genetic modification is uncertain and expensive.

These methods also have a high failure rate, which translates to a decrease in or even loss of enzyme activity in its entirety and more particularly on the substrate of interest. Such modifications therefore are no guarantee of a good selectivity.

There is consequently a need for a process for utilizing sulfides, especially those from mercaptan synthesis, that is viable industrially and economically.

There is more particularly a need for a process for oxidizing sulfides to sulfoxides that is applicable industrially, more economical and more environmentally friendly.

There is a need for a process for oxidizing sulfides to sulfoxides that is selective and that is simple and economical to operate industrially.

An objective of the present invention is to meet all or part of the needs above.

### SUMMARY OF THE INVENTION

The present invention hence relates to a process, preferably selective, for preparing a sulfoxide, comprising the following steps:

a) preparing a composition M comprising:
a sulfide;
an enzyme E catalyzing the oxidation of said sulfide to sulfoxide;
optionally at least one cofactor C of said enzyme E; and
an oxidant;

b) carrying out the enzymatic oxidation reaction of the sulfide to sulfoxide;

c) recovering the sulfoxide obtained in step b); and

d) optionally isolating and/or optionally purifying the sulfoxide recovered in step c);

wherein said sulfide is not entirely consumed during step b) of carrying out the enzymatic reaction.

### BRIEF DESCRIPTION OF THE DRAWINGS

The FIGURE represents the concentration of diethyl sulfide (DES), diethyl sulfoxide (DESO) and diethyl sulfone (DESO₂) present as a function of the time in a reaction catalyzed by the enzyme CHMO.

### DETAILED DESCRIPTION OF THE INVENTION

Surprisingly, the present inventors have found a selective process for preparing sulfoxides by enzymatic catalysis. With said process it is possible to obtain sulfoxides from the corresponding sulfides, more particularly without obtaining sulfones (or in negligible amount).

The reason is that the oxidation of sulfides by enzymatic catalysis takes place normally according to the following reaction sequence:

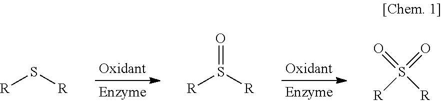

In the scheme above, the enzyme, any cofactor(s) thereof, and the oxidant used are the same in the first step, where the sulfoxide is formed, and in the second step, where the sulfone is formed. Within the same reaction mixture it is therefore possible to obtain both sulfoxides and sulfones, which is undesirable, as indicated above.

The present inventors have found a process allowing the sulfoxides to be obtained selectively by decreasing or even suppressing the by-products obtained and more particularly the sulfones. The inventors have thus determined the means of obtaining sulfoxides without obtaining sulfones (that is to say without the enzyme oxidizing the sulfoxides obtained to sulfones).

Surprisingly it has been found that the oxidation of sulfides to sulfoxides takes priority and occurs exclusively relative to the oxidation of the sulfoxides to sulfones. Accordingly, when there are sulfides in the reaction mixture (for example, in the composition M as defined above), the sulfoxides are formed selectively, without sulfones being formed. The sulfoxides are converted to sulfones when the reaction mixture (for example, the composition M as defined above) no longer contains any sulfides, but instead only sulfoxides.

Thanks to the invention it is thus possible to exert easy control over the production selectively of sulfoxides or of sulfones, and to do so without having to alter the operating conditions of the process.

### Definitions

The term “(C₁-C₂₀)alkyl” denotes saturated aliphatic hydrocarbons which may be linear or branched and which comprise from 1 to 20 carbon atoms. Preferably the alkyls comprise from 1 to 12 carbon atoms, or even from 1 to 4 carbon atoms. Examples include methyl, ethyl, n-propyl, isopropyl, n-butyl, isobutyl, sec-butyl and tert-butyl. The term “branched” is understood to mean that an alkyl group is substituted along the main alkyl chain.

The term “(C₂-C₂₀)alkenyl” denotes an alkyl as defined above that comprises at least one carbon-carbon double bond.

The term “(C₂-C₂₀)alkynyl” denotes an alkyl as defined above that comprises at least one carbon-carbon triple bond.

The term “(C₆-C₁₀)aryl” denotes monocyclic, bicyclic or tricyclic aromatic hydrocarbon compounds, more particularly phenyl and naphthyl.

The term “(C₃-C₁₀)cycloalkyl” denotes monocyclic or bicyclic saturated aliphatic hydrocarbons comprising from 3 to 10 carbon atoms, such as cyclopropyl, cyclobutyl, cyclopentyl or cyclohexyl.

(C₃-C₁₀)heterocycloalkane refers to a cycloalkane comprising from 3 to 10 carbon atoms and comprising at least one sulfur atom, preferably tetrahydrothiophene, and optionally at least one other heteroatom.

(C₄-C₁₀)heteroarene refers to an arene comprising between 4 and 10 carbon atoms and comprising at least one sulfur atom, for example, thiophene, and optionally at least one other heteroatom.

A heteroatom is understood in particular to be an atom selected from O, N, S, Si, P and halogens.

“Catalyst” is understood generally to be a substance which accelerates a reaction and which is unchanged at the end of this reaction. According to one embodiment, said enzyme E catalyzes the oxidation reaction of sulfides to sulfoxides.

A “catalytic amount” refers in particular to an amount sufficient to catalyze a reaction, more particularly to catalyze the oxidation of sulfides to sulfoxides. More particularly, a reagent used in a catalytic amount is used in a smaller amount, for example between around 0.01% and 20% by weight, relative to the amount by weight of a reagent used in stoichiometric proportion.

The selectivity of a reaction generally represents the number of moles of product formed, for example, the number of moles of sulfoxide formed, relative to the number of moles of reactant consumed following the reaction, for example, the number of moles of sulfide consumed.

The usual definitions of conversion, of selectivity and of yield are as follows:

Conversion=(number of moles of reactant in the initial state−number of moles of reactant remaining after the reaction)/(number of moles of reactant in the initial state)

Selectivity=Number of moles of reactant converted into the desired product/(number of moles of reactant in the initial state−number of moles of reactant remaining after the reaction)

Yield=conversion×selectivity

Hence a “selective process for preparing sulfoxides” refers especially to a process which consumes sulfides and produces sulfoxides, without sulfones being formed (or with a negligible amount of sulfones being formed). According to one embodiment, the oxidation reaction of the sulfides to sulfoxides is chemoselective.

For example, the process of the invention, more particularly step b), provides a selectivity of between 95% and 100%, preferably between 99% and 100% for the sulfoxides.

### Process

The process of the invention may be a selective and even chemoselective process for preparing sulfoxides. Said process preferably does not lead to the corresponding sulfones being formed.

According to one embodiment, it is step b), and more particularly the enzymatic oxidation reaction of the sulfides to sulfoxides carried out in step b), which is selective, preferably chemoselective.

According to one embodiment, said composition M always comprises an amount of sulfide which is sufficient for the enzyme E to convert the sulfide into sulfoxide, preferably without any sulfone being formed. According to one embodiment, the sulfide is provided in excess in the composition M.

The amount of sulfide remaining after step b), the step of carrying out the enzymatic reaction, may be between 0.0001% and 99.9% by weight, preferably between 0.1% and 99% by weight, preferably between 1% and 50% by weight, for example, between 1% and 10% by weight, relative to the starting amount of sulfide by weight, in other words the sulfide from step a).

More particularly, the composition M comprises:
a stoichiometric amount of a sulfide;
a catalytic amount of the enzyme E;
optionally a catalytic amount of at least one cofactor C; and
a sub-stoichiometric amount of the oxidant.

### Sulfide:

A sulfide is in particular an organic sulfide, this being any organic compound comprising at least one—C—S—C— function.

According to one embodiment, the composition M comprises at least one sulfide. It may for example comprise one, two or multiple different sulfides. Said sulfide may be symmetrical, meaning that the sulfur atom represents a center of symmetry relative to the compound.

According to one embodiment, said sulfide has the following general formula:

R₁—S—R₂ (I)

in which,

R₁ and R₂ may be identical or different and are selected, independently of one another, from the group consisting of:
(C₁-C₂₀)alkyl, (C₂-C₂₀)alkenyl, (C₂-C₂₀)alkynyl, (C₃-C₁₀)cycloalkyl and (C₆-C₁₀)aryl or

R₁ and R₂ form a ring with the sulfur atom to which they are attached, preferably a (C₃-C₁₀)heterocycloalkane or (C₆-C₁₀)heteroarene group;

it being possible for said alkyl, alkenyl, alkynyl, cycloalkyl, aryl, heterocycloalkane and heteroarene groups optionally to be substituted by one or more substituents;

and it being possible for said alkyl, alkenyl, alkynyl, cycloalkyl and aryl groups to comprise one or more heteroatoms.

Said alkyl, alkenyl, alkynyl, cycloalkyl, aryl, heterocycloalkane and heteroarene groups may optionally be substituted by one or more substituents selected from the group consisting of:
(C₁-C₂₀)alkyl, (C₃-C₁₀)cycloalkyl and (C₆-C₁₀)aryl;
   and may be optionally functionalized with one or more functions selected, without limitation and by way of example, from alcohol, aldehyde, ketone, acid, amide, nitrile and ester functions or else functions bearing sulfur, phosphorus and silicon.

According to one embodiment, said alkyl, alkenyl, alkynyl, cycloalkyl, aryl, heterocycloalkane and heteroarene groups may optionally be substituted by one or more substituents selected from the group consisting of: (C₁-C₂₀)alkyl, (C₃-C₁₀)cycloalkyl, (C₆-C₁₀)aryl, —OH, —C(O)OH, —C(O)H, —C(O)—NH₂, —NH₂, —NHR, —NRR′, —C(O)—, —C(O)—NHR′, —C(O)—NRR′, —COOR and —CN;

in which R and R′ represent, independently of one another, a (C₁-C₂₀)alkyl group.

According to one preferred embodiment, R₁ and R₂ may be identical or different and are selected, independently of one another, from the group consisting of:

(C₁-C₂₀)alkyl, (C₂-C₂₀)alkenyl, (C₂-C₂₀)alkynyl and (C₃-C₁₀)cycloalkyl or

R₁ and R₂ form, together with the sulfur atom to which they are attached, a (C₃-C₁₀)heterocycloalkane group.

R₁ and R₂ are preferably selected from (C₁-C₂₀)alkyls or R₁ and R₂ form, together with the sulfur atom bearing them, a (C₃-C₁₀)heterocycloalkane. The radicals R₁ and R₂ of said sulfide are preferably identical (i.e., so forming a symmetrical sulfide).

More preferably the sulfide is selected from dimethyl sulfide, diethyl sulfide, dipropyl sulfide, dibutyl sulfide, dioctyl sulfide, didodecyl sulfide and tetrahydrothiophene. Dimethyl sulfide is particularly preferred according to the invention. According to one embodiment, the sulfide is symmetrical and so is not prochiral. According to one embodiment, the sulfide is not tert-butyl methyl sulfide (CAS number 6163-64-0).

### Oxidant:

An oxidant is any compound that is able to oxidize a sulfide to sulfoxide.

The oxidant may be selected from the group consisting of air, oxygen-depleted air, oxygen-enriched air, pure oxygen and hydrogen peroxide. According to one particular embodiment, the oxidant is selected from the group consisting of air, oxygen-depleted air, oxygen-enriched air and pure oxygen when the enzyme E is a mono- or dioxygenase, and hydrogen peroxide when the enzyme E is a peroxidase. When the oxidant is in gaseous form it is present in the composition M as a dissolved gas. The percentage of oxygen in the enriched or depleted air is selected according to the reaction rate and the compatibility with the enzymatic system in a manner known to the skilled person.

The oxidant may be in a sub-stoichiometric amount in the composition M. Thus the sulfide present is partially consumed with the oxidant in the enzymatic reaction carried out in step b) but not entirely.

When air (air which may be depleted or enriched in oxygen) is used, it is obviously the oxygen within the air that is consumed during the enzymatic reaction carried out in step b) as oxidant.

At the end of said reaction, the oxygen is generally converted to water when the enzyme E used is a monooxygenase or consumed entirely when the enzyme E is a dioxygenase. The hydrogen peroxide in turn is converted to water subsequent to the action of the peroxidase. The process of the invention is therefore particularly advantageous in terms of emissions and environmental friendliness.

### Enzyme E:

Said enzyme E may be an oxidoreductase, preferably an oxidoreductase selected from the group consisting of monooxygenases, dioxygenases and peroxidases, more preferably from monooxygenases.

Said enzyme E is preferably a Baeyer-Villiger monooxygenase (BVMO).

More preferably still, and among the BVMOs, the enzyme E may be a cyclohexanone monooxygenase (CHMO), and more particularly a cyclohexanone 1,2-monooxygenase or a cyclopentanone monooxygenase (CPMO), and more particularly a cyclopentanone 1,2-monooxygenase.

The cyclohexanone 1,2-monooxygenases are in particular from class EC 1.14.13.22.

According to one particular embodiment, the CHMO is a CHMO from Acinetobactersp. (for example, of strain NCIMB 9871) and/or a CHMO encoded by the gene chnB belonging to cluster AB006902.

The cyclopentanone 1,2-monooxygenases are in particular from class EC 1.14.13.16.

According to one particular embodiment, the CPMO is a CPMO from Comamonas sp. (for example, the strain NCIMB 9872) and/or a CHMO encoded by the gene cpnB.

The monooxygenase may also be a hydroxyacetophenone monooxygenase (HAPMO) and more particularly a 4-hydroxyacetophenone monooxygenase.

The hydroxyacetophenone monooxygenases are in particular from class EC 1.14.13.84. According to one particular embodiment, the HAPMO is a HAPMO from Pseudomonas fluorescens that is encoded by the gene hapE.

### Cofactor(s) C:

“Cofactor C” refers especially to a cofactor needed for the catalytic activity of the enzyme E as defined above and/or allowing its catalytic activity to be enhanced.

According to one embodiment, one or two cofactors C or more are present in the composition M. For example, it is possible to admix the composition M with a cofactor C already present naturally in the enzyme E, in addition to another cofactor C.

When the oxidoreductase is a peroxidase, it is possible to add no cofactor C to the composition M. Nicotine and/or flavin cofactors may be used when the enzyme E belongs to the class of the mono- or dioxygenases.

Said at least one cofactor C may be selected from nicotine cofactors and flavin cofactors. More particularly, said at least one cofactor C may be selected from the group consisting of: nicotinamide adenine dinucleotide (NAD), nicotinamide adenine dinucleotide phosphate (NADP), flavin mononucleotide (FMN), flavin adenine dinucleotide (FAD) and/or the corresponding reduced form thereof (that is, NADH, H+ NADPH, H+, FMNH₂, FADH₂).

The cofactors C listed above are advantageously used in their reduced forms (for example, NADPH, H+) and/or their oxidized forms (for example, NADP+), meaning that they can be added in these reduced and/or oxidized forms to the composition M.

The enzyme E used is preferably cyclohexanone monooxygenase, for example, the cyclohexanone monooxygenase from Acinetobacter sp., and the cofactor C used is NADP, optionally supplemented by FAD.

### System(s) for Regenerating the Cofactor(s) C:

The composition M as defined above may also comprise at least one system for regenerating the cofactor(s) C. A “system for regenerating the cofactor(s) C” means any chemical and/or enzymatic reaction or suite of reactions allowing the reduced cofactor(s) C to be reconverted into oxidized cofactor(s) C or vice versa.

The regeneration systems may for example be known enzymatic redox systems, with use of a sacrificial substrate. Systems of this kind involve the use of a second enzyme (called the recycling enzyme) that enables recycling of the cofactor(s) C used, by using a sacrificial substrate.

Recycling enzymes include glucose dehydrogenase, dehydrogenase formate, phosphite dehydrogenase (Vrtis, Angew. Chem. Int. Ed., 2002, 41(17), 3257-3259) or else dehydrogenase alcohols (Leuchs, Chem. Biochem. Eng. Q., 2011, 25(2), 267-281; Goldber, App. Microbiol. Biotechnol., 2007, 76(2), 237).

Among the sacrificial substrates which may be used within the context of the present invention, hydrogen-donating compounds are most particularly preferred, and among these, the entirely suitable compounds are hydrogen-donating organic reducing compounds bearing a hydroxyl function, such as alcohols, polyols, sugars, etc., such as glucose or glycerol.

For example, in the case of CHMO, the enzyme of the recycling system reduces the cofactor NADP+ in the form NADPH, H+, with the sacrificial substrate being oxidized.

The composition M according to the invention may also comprise:
optionally one or more solvents chosen from water, buffers such as phosphate buffers, Tris-HCl, Tris base, ammonium bicarbonate, ammonium acetate, HEPES (4-(2-hydroxyethyl)-1-piperazineethanesulfonic acid), CHES (N-cyclohexyl-2-aminoethanesulfonic acid), or salts such as sodium chloride, potassium chloride, or mixtures thereof;
optionally additives such as surfactants, in order in particular to promote the solubility of one or more reactants or substrates of the enzymatic reaction.

Preferably, the composition M is an aqueous solution. For example, said composition M comprises between 50% and 99% by weight of water, preferably between 80% and 97% by weight of water, relative to the total weight of the composition M.

According to one embodiment, the composition M is deemed to comprise the reaction mixture.

The various components of the composition M prepared in step a) above are readily obtainable commercially or may be prepared by techniques well known to the skilled person. These different elements may be in solid, liquid or gaseous form and may very advantageously be rendered into solution or dissolved in water or any other solvent to be used in the process of the invention. The enzymes used may also be grafted onto a support (in the case of supported enzymes).

According to one embodiment, the enzyme E, optionally said at least one cofactor C, optionally said at least one regeneration system are present:
in isolated and/or purified form, for example, in aqueous solution;
or in a crude extract, i.e., in an extract of milled cells; or
in whole cells.

Whole cells are used with preference. The ratio [sulfide] (in mmol/L)/[cells] (in g_{cdw}·L⁻¹) may be between 0.01 and 10, preferably between 0.01 and 3 mmol/g_{cdw}, preferably during step b), the step of carrying out the enzymatic reaction. The concentration by mass in grams of dry cells (g_{CDW} for Cells Dry Weight) is determined by conventional techniques.

The enzyme E may or may not be overexpressed in said cells, which are referred to below as host cells.

The host cell may be any host appropriate for producing an enzyme E from the expression of the corresponding coding gene. This gene will then be either located in the genome of the host or carried by an expression vector such as those defined below.

For the purposes of the present invention, “host cell” is in particular understood to be a prokaryotic or eukaryotic cell. Host cells commonly used for the expression of recombinant or non-recombinant proteins include in particular cells of bacteria such as Escherichia coli or Bacillus sp., or Pseudomonas, cells of yeasts such as Saccharomyces cerevisiae or Pichia pastoris, cells of fungi such as Aspergillus niger, Penicillium funiculosum or Trichoderma reesei, insect cells such as Sf9 cells, or else mammalian (in particular, human) cells such as the HEK 293, PER-C6 or CHO cell lines.

Said host cells may be in stationary growth phase, having been removed from the culture medium, for example.

Preferably, the enzyme E and its at least one cofactor C where relevant are expressed in the bacterium Escherichia coli. The CHMO is preferably expressed within a strain of Escherichia coli such as for example Escherichia coli BL21(DE3).

The HAPMO is preferably expressed within a strain of Escherichia coli such as for example Escherichia coli BL21(DE3).

In the case of whole and/or lyzed cells, for example, it is the cellular machinery that regenerates the cofactor(s) C used. For example, in the case of an Escherichia coli strain which expresses cyclohexanone monooxygenase (CHMO), the cofactor C is NADP.

According to one embodiment, when the CHMO converts the sulfide into sulfoxide, the cofactor C₁ is NADP, optionally with the cofactor C2 FAD. When the CHMO converts the sulfide into sulfoxide, the cofactor NADPH, H⁺ is oxidized to NADP⁺, which will be regenerated by the cell and/or by the regeneration system installed. Regeneration of the reduced cofactor will be enabled by enzymes naturally present in E. coli, particularly the enzyme glycerol dehydrogenase, if the medium is supplemented with glycerol, for example. In the case of a medium supplemented with glucose, the enzymes of the pentose phosphate pathway, and especially the enzymes glucose-6-phosphate dehydrogenase and/or 6-phosphogluconic acid dehydrogenase, which are naturally present in E. coli, will participate in the regeneration of the reduced cofactor C1.

According to the invention, the host cell comprising the enzyme E, optionally at least one cofactor C and optionally a system for regenerating the cofactor(s) C is referred to as a “biocatalyst”.

The enzyme E and/or biocatalyst as defined above may be obtained by various techniques known to the skilled person.

### Integration of an Expression Vector Comprising the Coding Sequence for the Enzyme E in the Cellular Host

When an expression vector such as a plasmid is used, transformation of the prokaryotic and eukaryotic cells is a technique well known to the skilled person, as for example by lipofection, electroporation, heat shock, or by chemical methods. The expression vector and the method of introducing the expression vector within the host cell are chosen according to the host cell selected. This transformation step yields a transformed cell that expresses a gene coding for a recombinant enzyme E. The cell may be cultivated, in a culturing/incubating step, to produce the enzyme E.

The incubation/culturing of prokaryotic and eukaryotic cells is a technique well known to the skilled person, who is able to determine, for example, the culture medium or else the temperature and time conditions. Depending on the vector used, an induction period—corresponding to increased production of the enzyme E—may be observed. Consideration may be given to using a weak (as for example arabinose for the vector pBad) or strong (as for example isopropyl β-D-1-thiogalactoside (IPTG) for the vectors pET22b, pRSF, etc.) inductor. Production of the enzyme E by the host cell may be verified using the technique of SDS-PAGE electrophoresis or the Western blot technique.

An “expression vector” is a DNA molecule of reduced size into which a nucleotide sequence of interest can be inserted. Selection may be made from a number of known expression vectors, such as plasmids, cosmids, phages, etc.

The vector is selected particularly as a function of the cellular host that is used.

The expression vector in question may be, for example, that described in document WO 83/004261.

### Integration of the Coding Sequence for the Enzyme E in the Genome of the Host Cell in the Absence of an Expression Vector

The nucleotide sequence coding for the enzyme E may be integrated into the genome of the host cell by any known method, such as, for example, by homologous recombination or else by the system CRISPR-Cas9 etc. Production of the enzyme E by the host cell may be verified using the technique of SDS-PAGE electrophoresis or the Western blot technique.

Isolation and/or Purification of the Enzyme E for Use in Isolated and/or Purified Form

Following transformation and culturing/incubation of the transformed host cell, a step of isolation and optionally of purification of the enzyme E may be carried out. In this way, the process of the invention is carried out not in the presence of the host cells but by the enzyme E in solution in the composition M, preferably in aqueous solution.

The isolation and/or the purification of said enzyme E produced may be carried out by any means known to the skilled person. This may for example involve a technique selected from electrophoresis, molecular sieving, ultracentrifugation, differential precipitation, for example with ammonium sulfate, ultrafiltration, membrane or gel filtration, ion exchange, separation via hydrophobic interactions, or affinity chromatography, such as IMAC, for example.

### Mode of Lysis of the Host Cell, Preparation of a Crude Extract of Milled Cells

The cell lysate may be obtained by various known techniques such as sonication, pressure (French press), via the use of chemical agents (e.g., Triton), etc. The lysate obtained corresponds to a crude extract of milled cells.

In step a), the various components of the composition M may be added in any desired order. The composition M may be prepared by simply mixing the various components.

According to one embodiment, the process of the invention comprises a step b′), between step b) and step c), in which the enzymatic reaction is halted by inactivation of the biocatalyst and/or of the enzyme E. This step b′) may be carried out by known means such as heat shock (for example, with a temperature of around 100° C.) or osmotic shock, application of a high pressure, addition of a solvent enabling destruction and/or precipitation of the cells and/or the enzymes E, pH modification (either a low pH of around 2, or a high pH of around 10).

The sulfide may be introduced into the composition M at a rate higher than the reaction rate in the enzymatic reaction according to step b).

According to one embodiment, step b), the step of carrying out the enzymatic reaction, is carried out at a pH of between 4 and 10, preferably between 6 and 8 and more preferably between 7 and 8—for example, 7.

According to one embodiment, step b), the step of carrying out the enzymatic reaction, is carried out at a temperature of between 5° C. and 100° C., preferably between 20° C. and 80° C. and more preferably between 25° C. and 40° C.

The pressure used for said enzymatic reaction may range from a reduced pressure compared to atmospheric pressure to several bar (several hundred kPa), depending on the reactants and equipment used.

The advantages procured by the process of the invention are many. These advantages include the possibility of working in aqueous solution, under very mild temperature and pressure conditions and under pH conditions close to neutrality. All of these conditions are typical of a biocatalytic process referred to as being “green” or “sustainable”.

In step c), the sulfoxide may be recovered in liquid or solid form. The sulfoxide may be recovered in aqueous solution, or in liquid form by decanting, or even in solid form by precipitation, depending on its solubility.

For step d), the methods of purification are dependent on the characteristics of the sulfoxide in question. Accordingly, after separation of the cells (containing the enzyme E) by ultrafiltration or centrifugation, distillation may enable separation of the sulfoxide.

This distillation may take place at atmospheric pressure, reduced pressure (vacuum), or under higher pressure if the skilled person deems it to hold any advantage.

Membrane separation may also be contemplated for the purpose of reducing the water content of the mixture for distillation, or of accelerating a crystallization process. If the sulfoxide has been recovered by decanting from an aqueous reaction mixture, drying over molecular sieve (or any other drying method) may be contemplated.

Said process may be carried out batchwise or continuously.

The process of the invention may comprise the following steps:
a 1) preparing a composition comprising:
   said enzyme E;
   optionally said at least one cofactor C;
   said oxidant;
a 2) preparing the composition M as defined above by adding said sulfide, preferably by injection, to the composition obtained in step a-1);
b) carrying out the enzymatic oxidation reaction of the sulfide to sulfoxide;
c) recovering the sulfoxide obtained in step b); and
d) optionally isolating and/or optionally purifying the sulfoxide recovered in step c).

According to another implementation, the process may contain the following steps:
a 1) preparing a composition comprising:
   said sulfide;
   said enzyme E; and
   optionally said at least one cofactor C;
a 2) preparing the composition M as defined above by adding said oxidant to the composition obtained in step a-1);
b) carrying out the enzymatic oxidation reaction of the sulfide to sulfoxide;
c) recovering the sulfoxide obtained in step b); and
d) optionally isolating and/or optionally purifying the sulfoxide recovered in step c).

### Composition M

The present invention also relates to the composition M as defined above.

The various elements of the composition M, as such and for the uses thereof, are as defined for the process above.

More particularly, the present invention also relates to a composition M comprising:
a symmetrical sulfide as defined above;
an oxidoreductase enzyme as defined above, preferably a Baeyer-Villiger monooxygenase (BVMO), more preferably a cyclohexanone monooxygenase (CHMO), catalyzing the oxidation of said symmetrical sulfide to symmetrical sulfoxide;
optionally at least one cofactor C of said enzyme E as defined above; and
optionally an oxidant as defined above.

More particularly still, the present invention relates to a composition M comprising:
a sulfide of the following general formula (I): R₁—S—R₂ (I) in which R₁ and R₂ are identical and as defined above;
an oxidoreductase enzyme as defined above, preferably a Baeyer-Villiger monooxygenase (BVMO), more preferably a cyclohexanone monooxygenase (CHMO), catalyzing the oxidation of said sulfide (I) to sulfoxide of the following general formula (II): R₁—S(O)—R₂ (II)

in which R₁ and R₂ are identical and as defined above;
optionally at least one cofactor C of said enzyme E as defined above; and
optionally an oxidant as defined above.

The sulfide is preferably selected from dimethyl sulfide, diethyl sulfide, dipropyl sulfide, dibutyl sulfide, dioctyl sulfide, didodecyl sulfide and tetrahydrothiophene. Dimethyl sulfide is an especially preferred sulfide.

According to one embodiment, said composition corresponds to the composition M as defined above, for implementing the process as defined above.

### Uses

The present invention also relates to the use of an oxidoreductase enzyme, preferably a Baeyer-Villiger monooxygenase (BVMO), more preferably a cyclohexanone monooxygenase (CHMO) as defined above, for oxidizing a symmetrical sulfide to the corresponding symmetrical sulfoxide. Said enzymatic oxidation reaction is, in particular, selective in the sense of the invention. According to one embodiment, the sulfide is of general formula R₁—S—R₂ (I) and is converted into a sulfoxide of general formula R₁—S(O)—R₂ (II), in which R₁ and R₂ are identical and as defined above.

The FIGURE represents the concentration (in mM) of diethyl sulfide (DES), diethyl sulfoxide (DESO) and diethyl sulfone (DESO₂) present in the reaction mixture as a function of the time (in hours), when the reaction is catalyzed by the enzyme CHMO.

The expression “between X and X” includes the stated endpoints. The examples below are given for illustrative purposes and do not limit the present invention.

### EXAMPLES

### Example 1: Selective Synthesis of Diethyl Sulfoxide from Diethyl Sulfide According to the Invention

I. Preparation of the Biocatalyst:

A strain of Escherichia coli BL21(DE3) (sold by Merck Millipore) expressing the chnB gene inserted into the plasmid pET22b (sold by Promega, Qiagen) was constructed. It enables the heterologous expression of CycloHexanone MonoOxygenase (CHMO) from Acinetobacter sp.

It will be appreciated that said strain comprises the CHMO, the cofactors of the CHMO, namely NADP and FAD, and its regeneration system.

This strain was precultured and cultured by the techniques known to the skilled person.

After an induction phase triggered by adding isopropyl β-D-1-thiogalactoside (IPTG) at a final concentration of 0.85 mmol/L, a certain volume of the culture is centrifuged (10 min, 5000 g, 4° C.) to give the desired amount of cells. In this example, a pellet of 300 ODU of fresh cells is then resuspended in 32 mL of a 0.1 mol/L phosphate buffer at pH 7 supplemented with 5 g/L of glycerol. The cell concentration then obtained is 9.4 ODU/mL or else 3 g_{CDW}/L (where CDW stands for cells dry weight).

II. Bioconversion:

In a 250 mL flask containing 32 mL of the mixture described above, the initial concentration of diethyl sulfide (DES) is measured at 4.5 mmol/L at t=0.

At regular intervals, 50 μL of the reaction mixture are withdrawn and diluted in 1450 μL of an acetonitrile solution containing 25 mg/L of undecane (internal standard). After centrifugation (5 min, 12 500 g), the supernatant is injected in GC (gas chromatography) for quantitative measurement of the diethyl sulfoxide (DESO) and diethyl sulfone (DESO₂) formed during the reaction. Under the conditions of the analysis performed, the minimum measurable concentration is 30 μM.

The analyses show a change in chemoselectivity at 2.5 h of reaction. Before this point, a linear increase in the amount of DESO is measured, with no sulfone being detected. The rate of oxidation of the sulfide over this part is then 4 mmol of DES oxidized per liter of mixture per hour.

After 3 h, a linear increase in the amount of DESO₂ is observed at the same time as the consumption of the DESO, with the DESO₂ being formed at a rate of 2.8 mmol/L/h. At 6 h of reaction, only the DESO₂ is present.

DESO₂ is therefore formed when DES is no longer detected in the mixture.

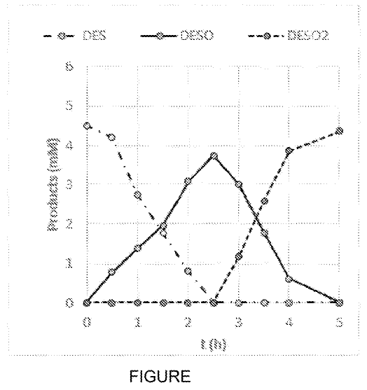

This example shows that the oxidation reaction of the sulfides to sulfoxides is chemoselective while the sulfide is present in the reaction mixture (cf. FIG. 1).

The selectivity obtained is around 100%.

Firstly, when the DES is still present in the reaction mixture, the sulfone is not detected with the analytical tools used.

Secondly, when the DES is no longer detected in the reaction mixture, the oxidation of the DESO is complete. At the end of the reaction (t=5 h), around 100% of DESO₂ is obtained, with no DESO being detected by the analytical tools used.

By extrapolation, the ratio [sulfide] (in mmol/L)/[cells] (in gcdw·L−1) calculated is 0.06 mmol/gcdw at t=2.5.

### Example 2

I. Preparation of the Biocatalyst:

The same strain as that described in example 1 was used in this example.

This time a larger amount of cells was used.

At the end of the induction step, the OD₆₀₀ is measured at 8.4 ODU/mL and a volume of 102 mL is withdrawn, to give, after centrifugation (10 min, 5000 g, 4° C.), a pellet containing 860 ODU of fresh cells. This pellet is then resuspended in 32 mL of a 0.1 mol/L phosphate buffer at pH 7 supplemented with 0.5 g/L of glycerol. The cell concentration then obtained is 27 ODU/mL (or around 9 g_{CDW}/L).

II. Bioconversion:

In a 250 mL flask containing 32 mL of the mixture described above, the concentration of diethyl sulfoxide (DESO) measured in the reaction mixture is 11.3 mmol/L. At t=2 h, DES in ethanolic solution is added: a concentration of DES of 10.4 mmol/L is then measured.

The reaction is monitored by performing the two sampling operations described in example 1. The analyses show that between 0 and 2 h of reaction, only DESO₂ is produced from the DESO added initially (a concentration of 7.7 mmol/L is then obtained). Following the addition of DES at t=2 h, the concentration of DESO₂ no longer varies up to at least 4.5 h, whereas in the same time DESO is produced (10.4 mmol/L are produced). At 16.5 h of reaction, only DESO₂ is present (the DESO having completely oxidized).

Adding DES therefore interrupts the formation of the sulfone (in this case, DESO₂).

This example shows that the oxidation of the sulfide to sulfoxide is not only the priority reaction but is also exclusive in relation to the oxidation reaction of the sulfoxide to sulfone.

### Example 3: Enzymatic Oxidation of Dimethyl Sulfide (DMS)

I. Preparation of the Biocatalyst:

The biocatalyst (CHMO) is identical to that in example 1 and is produced under the conditions described in said example 1.

II. Bioconversion:

The bioconversion conditions presented in example 1 are identical to those used for this example, except for the sulfide used. In this example, an ethanolic solution of DMS is used to obtain an initial sulfide concentration of 4.5 mM.

The reaction is monitored by the method presented in example 1.

Surprisingly, the biocatalyst used results in the same oxidation characteristics. Specifically, the DMS is oxidized chemoselectively when it is present in the mixture (no dimethyl sulfone detected), and then the sulfoxide is oxidized from the time at which DMS is no longer detected in the mixture.

Besides this chemoselectivity, a sulfide oxidation rate of the same order of magnitude (relative to the DES) was obtained in the early stages of reaction: 3.9 mmol of DMS are oxidized per liter of mixture per hour. The sulfone formation rate is 1 mmol/L/h, moreover.

### Example 4: Enzymatic Oxidation of Methyl Ethyl Sulfide (MES)

I. Preparation of the Biocatalyst:

The biocatalyst (CHMO) is identical to that in example 1 and is produced under the conditions described in said example 1.

II. Bioconversion:

The bioconversion conditions presented in example 1 are identical to those used for this example, except for the sulfide used. In this example, an ethanolic solution of MES is used to obtain an initial sulfide concentration of 4.5 mM.

The reaction is monitored by the method presented in example 1.

Surprisingly, the biocatalyst used results in the same oxidation characteristics. Specifically, the MES is oxidized chemoselectively when it is present in the mixture (no methyl ethyl sulfone detected), and then the methyl ethyl sulfoxide is oxidized from the time at which MES is no longer detected in the mixture.

Besides this chemoselectivity, an oxidation rate of the same order of magnitude (relative to the other sulfides) was obtained in the early stages of reaction: 3.6 mmol of MES are oxidized per liter of mixture per hour. The sulfone production rate is 3.5 mmol/L/h, moreover.

### Example 5: Enzymatic Oxidation of Tetrahydrothiophene (THT)

I. Preparation of the Biocatalyst:

The biocatalyst (CHMO) is identical to that in example 1 and is produced under the conditions described in said example 1.

II. Bioconversion:

The bioconversion conditions presented in example 1 are identical to those used for this example, except for the sulfide used. In this example, an ethanolic solution of THT is used to obtain an initial sulfide concentration of 4.5 mM.

The reaction is monitored by the method presented in example 1.

Surprisingly, the biocatalyst used results in the same oxidation characteristics. Specifically, the THT is oxidized chemoselectively when it is present in the mixture (no sulfolane is detected, which is the corresponding sulfone), and then the tetrahydrothiophene 1-oxide is oxidized from the time at which THT is no longer detected in the mixture.

Besides this chemoselectivity, an oxidation rate of the same order of magnitude (relative to the other sulfides) was obtained in the early stages of reaction: 3.4 mmol of THT are oxidized per liter of mixture per hour. The sulfone formation rate is 1.5 mmol/L/h, moreover.

### Example 6: Enzymatic Oxidation of a Mixture of Sulfides According to the Invention

I. Preparation of the Biocatalyst:

The same strain as that described in example 1 was used in this example.

At the end of the induction step, the OD₆₀₀ is measured at 8.4 ODU/mL and a volume of 31 mL is withdrawn, to give, after centrifugation (10 min, 5000 g, 4° C.), a pellet containing 300 ODU of fresh cells. This pellet is then resuspended in 32 mL of a 0.1 mol/L phosphate buffer at pH 7 supplemented with 0.5 g/L of glycerol. The cell concentration then obtained is 9.4 ODU/mL (or around 3 g_{CDW}/L).

II. Bioconversion:

In a 250 mL flask containing 32 mL of the mixture described above, 75 μL of ethanolic solutions of diethyl sulfide (DES), dimethyl sulfide (DMS) and tetrahydrothiophene (THT) each at 3.64 M are introduced at the same time: this is the start of the reaction.

The reaction is monitored by performing the same sampling operation described in example 1.

The analyses show that during a first period, the sulfides in the mixture are oxidized to sulfoxides, with no sulfones being detected. Surprisingly, the rate of oxidation of the DES is greater than those of the other two sulfides present (DMS and THT), both of which have the same oxidation rate (cf. table 1 below).

TABLE 1

| Substrate | DMS | DES | THT |
|---|---|---|---|
| Sulfoxide formation rate (mmol/L/h) | 0.5 | 2.5 | 0.55 |
| Sulfone formation rate (mmol/L/h) | — | 1.5 | 0.6 |

Only in a second phase, when the sulfides are no longer detected in the reaction mixture, are the corresponding sulfones obtained, with the exception of DMSO₂, which was not detected under these conditions with mixing of the different sulfides.

1. A process for preparing a sulfoxide, comprising:
a) preparing a composition M comprising:
a sulfide;
an enzyme E catalyzing oxidation of said sulfide to sulfoxide;
optionally at least one cofactor C of said enzyme E; and
an oxidant;
b) carrying out the enzymatic oxidation reaction of the sulfide to sulfoxide;
c) recovering the sulfoxide obtained in step b); and
d) optionally isolating and/or optionally purifying the sulfoxide recovered in step c);
wherein said sulfide is not entirely consumed during step b) of carrying out the enzymatic reaction.

2. The process as claimed in claim 1, wherein said enzyme E is an oxidoreductase.

3. The process as claimed in claim 1, wherein said enzyme E is a Baeyer-Villiger monooxygenase (BVMO).

4. The process as claimed in claim 1, wherein said sulfide is of the following general formula:
R₁—S—R₂ (I)
wherein:
R₁ and R₂ may be identical or different and are selected, independently of one another, from the group consisting of:
(C₁-C₂₀)alkyl, (C₂-C₂₀)alkenyl, (C₂-C₂₀)alkynyl, (C₃-C₁₀)cycloalkyl and (C₆-C₁₀)aryl or
R₁ and R₂ form, together with the sulfur atom to which they are attached, a heterocycloalkane or a heteroarene;
where said alkyl, alkenyl, alkynyl, cycloalkyl, aryl, heterocycloalkane and heteroarene groups are optionally substituted by one or more substituents;
where said alkyl, alkenyl, alkynyl, cycloalkyl and aryl groups optionally comprise one or more heteroatoms.

5. The process as claimed in claim 4, wherein the radicals R₁ and R₂ of said sulfide are identical.

6. The process as claimed in claim 1, wherein said sulfide is selected from dimethyl sulfide, diethyl sulfide, dipropyl sulfide, dibutyl sulfide, dioctyl sulfide, didodecyl sulfide and tetrahydrothiophene.

7. The process as claimed in claim 1, wherein the ratio [sulfide] (in mmol/L)/[cells] (in g_{cdw}·L⁻¹) is between 0.01 and 10 mmol/g_{cdw}.

8. The process as claimed in claim 1, wherein the oxidant is selected from the group consisting of air, oxygen-depleted air, oxygen-enriched air, pure oxygen and hydrogen peroxide.

9. The process as claimed in claim 1, wherein the cofactor C is selected from nicotine cofactors and flavin cofactors.

10. The process as claimed in claim 1, wherein the enzyme E is cyclohexanone monooxygenase and the cofactor C is NADP and optionally FAD.

11. A composition comprising:
a symmetrical sulfide;
an oxidoreductase enzyme catalyzing oxidation of said symmetrical sulfide to symmetrical sulfoxide;
optionally at least one cofactor C of said enzyme E; and
optionally an oxidant.

12. The composition as claimed in claim 11, wherein the sulfide (I) is selected from dimethyl sulfide, diethyl sulfide, dipropyl sulfide, dibutyl sulfide, dioctyl sulfide, didodecyl sulfide and tetrahydrothiophene.

13. A method for oxidizing a symmetrical sulfide to symmetrical sulfoxide, the method comprising reacting the sulfide with an oxidoreductase enzyme.
